(19) United States
(12) Patent Application Publication
(10) Pub. No.: US 2009/0099421 A1
(43) Pub. Date: Apr. 16, 2009

(54) Intubation laryngoscope with a double holder

(75) Inventors: Micheal Shalman, Ashdod (IL); Leonid Lukov, Beer-sheva (IL)

Correspondence Address: NIXON & VANDERHYE, PC, 901 NORTH GLEBE ROAD, 11TH FLOOR, ARLINGTON, VA, 22203, US

(73) Assignee: M.S.VISION LTD, Ashkelon (IL)

(21) Appl. No.: 11/907,451

(22) Filed: Oct. 12, 2007

(51) Int. Cl. A61B 1/267

(52) U.S. Cl. 600/197; 600/199

(57) ABSTRACT

Intubation laryngoscope having reusable and disposable blades, an illumination system, and an advantageous fastening appliance for connecting the blades to the laryngoscope handle.

### BACKGROUND

1. Field of the Invention

The present invention relates to the intubation laryngoscopes having reusable and disposable blades, an illumination system, and an improved fastening appliance for connecting the blades to the laryngoscope handle.

2. Prior Art

The most widely spread design of intubation laryngoscope 100, for example fabricated by Welch Allyn company (see enclosed FIGS. 1 to 3), comprises a blade set, including four detachable blades of curvilinear Macintosh profile of various sizes designed for operations with children and adults. Each blade 101, made of stainless steel, extends from a lower end of handle 104 in a distal direction and has blade distal end portion 102 designed to expose a tracheal entrance in order to insert an endotracheal tube therein, as well as blade proximal end portion 103 designed for detachable connection with laryngoscope handle 104 through blade holder 105. The latter consists of two parts including upper holder part 106 immovably affixed to a lower end of handle 104, and lower holder part 107 immovably fastened to blade proximal end portion 103 and movably fastened to upper holder part 106 by a holder fastening means. The latter include: two lateral walls 108, 109 of upper holder part 106 spaced some distance apart, protruding downward and arranged in parallel with a plane passing through the distal direction of the detachable blade extension (FIG. 3) and a longitudinal axis of the handle; axle 110 mounted between lateral walls 108, 109 at a distal edge of upper holder part 106; insertion portion 111 of lower holder part 107 to be inserted between lateral walls 108, 109 with a small gap; hook 112 in the form of straight-line groove 113, opened from a distal lateral side, disposed at a distal edge of insertion portion 111 and adapted for putting on axle 110 to envelop the axle from upper, lower and proximal sides with a capability of rotating lower holder part 107 along with blade 101 around axle 110 clockwise in order to set lower holder part 107 into an operation position shown in FIG. 3 and allowing an intubation execution, or counter-clockwise in order to set lower holder part 107 into inoperative position, specifically to detach blade 101 along with lower holder part 107 from handle 104.

The holder fastening means also comprise a holder locking means preventing lower holder part 107 from spontaneous dislocation relative to upper holder part 106 in the operation position and, specifically, including a detent means preventing lower holder part 107 from spontaneous rotation around axle 110 in the operation position. The detent means comprise detent recess 114 located on an inner side of lateral wall 108, and ball springy detent 115 mounted at insertion portion 111 oppositely to the hook disposition and having a ball adapted to enter detent recess 114 in the operation position and to come out from the detent recess in the inoperative position. The holder locking means also include a keeping means made as ball keeper 116 mounted in a lower wall of hook groove 113, interacting with axle 110 and preventing lower holder part 107 from spontaneous dislocating relative to upper holder part 107 in the operation position and from spontaneous falling out of upper holder part 106 in the inoperative position. Mounting the ball keeper 116 is implemented through upper window 119, which substantially weakens hook 112.

Laryngoscope 100 is provided with an illumination means designed to illuminate a zone of endotracheal tube insertion in patient's mouth and comprising batteries housed in the handle, a light source disposed in upper holder part 106, light guide 117 located in a unit of lower holder part 107 and blade 101, and a switch for turning on/of an illumination light. The latter includes movable element 118 disposed on upper holder part 106 between lateral walls 108, 109 at some distance from axle 110, protruding downward from an inner upper surface of upper holder part 106, and interacting with lower holder part 107 so that, in the operation position of the lower holder part, movable element 118 is dislocated upward by pressure of lower holder part 107 thereby turning on the illumination light. In the inoperative position the movable element is released by the lower holder part that leads to returning downward the movable element and thereby to turning off the illumination light.

In version embodiment of intubation laryngoscopes, fabricated by company “Penlon”, the blade is made of plastic and the locking means are made as latches including plastic projections located on resilient legs arranged on the holder lower part.

In another version embodiment, fabricated by several companies, specifically by “Ismel Ltd”, the illumination means include a light source disposed on the blade and connected with the batteries housed in the handle through the switch identical to described above.

There is a host of patents, devoted to the solutions of various laryngoscope problems, wherein the laryngoscopes identical to above are presented, for example, U.S. Pat. No. 4,579,108, U.S. Pat. No. 6,454,704, U.S. Pat. No. 5,003,962, U.S. Pat. No. 5,800,344, U.S. Pat. No. 5,575,758, US 2004/0242967A1, US 2007/0093693 A1.

The disadvantage of the above intubation laryngoscope is its relatively high cost and complexity caused with the necessity to provide each blade of the blade set with its own lower holder part.

Another disadvantage is the need for special ball keeper to prevent the lower holder part from the lateral dislocation in the operation position and from spontaneous falling out from the upper holder part in the inoperative position, resulting in the design complication, rise in price, and weakening the hook strength.

Another disadvantage is the application restrictions of laryngoscope design, specifically, eliminating the capability of the updated blade using without the handle refinement.

Another disadvantage is the load distribution characterized by excessive loading the hook resulting in insufficient hook strength in the version of blade and lower holder part made of plastic.

Another disadvantage is the necessity for arrangement of parts of the illumination system, such as the light guide or the light source, in each blade, leading to increasing the price of the blades and the illumination system.

### SUMMARY OF THE INVENTION

The objective of the present invention is substantial decrease in price of laryngoscope due to the capability of using a common single lower holder part for each blade of the blade set.

Another objective is decrease in price of laryngoscope as a result of the elimination of the ball keeper due to fulfilling the ball keeper functions by the other elements of the lower holder part.

Another objective is substantial reducing the requirements for the strength of the lower holder part, specifically in the plastic embodiment, due to the redistribution of loading the elements of the lower holder part.

Another objective is the extension of the laryngoscope applicability by updating only its lower holder part resulting in the capability of using the blade various designs.

Another objective is simplification and decreasing in the price of the detachable blade as a result of eliminating the illumination system parts from the blade design.

The above noted objectives are accomplished with an intubation laryngoscope comprising a handle and at least one detachable blade extending from a lower end of said handle in a distal direction and having a blade distal end portion designed to expose a tracheal entrance in order to insert an endothracheal tube therein and a blade proximal end portion. There is also a holder designed for fastening the blade proximal end portion to the handle lower end and including an upper holder part immovably affixed to a lower end of said handle, and a lower holder part movably fastened to the upper holder part and adapted for detachable connection with the blade proximal end portion.

Such lower holder part is a novelty allowing using a common single lower holder part for each blade of the blade set, thereby providing decrease in price of laryngoscope in comparison with the known versions having the lower holder part on each blade of blade set. Moreover, there is a capability of the easy laryngoscope adaptation to operation with various blade kinds by changing only the single lower holder part.

The offered lower holder part is detachably fastened to the upper holder part by a holder fastening means including: two lateral walls of the upper holder part spaced some distance apart and protruding downward; an axle mounted between the lateral walls at an edge of the upper holder part; an insertion portion of the lower holder part to be inserted between the lateral walls with a small gap; a hook disposed at an edge of the insertion portion of lower holder part and adapted to putting on the axle with a capability of rotating the lower holder part around the axle to set a needed position of the lower holder part relative to the upper holder part; a holder locking means preventing the lower holder part from spontaneous dislocation relative to the upper holder part in the operation position allowing the intubation execution.

The intubation laryngoscope comprises an illumination means designed to illuminate the patient's mouth zone of the endotracheal tube insertion, and provided with a light source and a switch for turning on/off an illumination light. The switch includes a movable element disposed on the upper holder part between the lateral walls at some distance from the axle, protruding downward from an inner upper surface of the upper holder part, and interacting with the lower holder part. In the operation position of the lower holder part the movable element is dislocated upward by pressure of the lower holder part thereby turning on the illumination light. In the inoperative position the movable element is released by the lower holder part that leads to returning downward the movable element and thereby to turning off the illumination light.

The mentioned hook is formed as a groove having a U-shaped upper portion directed downward with its open side so that inner walls of the U-shaped upper portion are disposed substantially in parallel with a longitudinal axis of the handle and interact with the axle thereby preventing the lower holder part from dislocating in a lateral directions relative to the upper holder part in the operation position. Such arrangement of the hook groove is novelty allowing the groove inner walls to fulfill the function of the ball keeper. As a result, the ball keeper and the upper window used for the ball keeper mounting may be eliminated that reduces the laryngoscope price and strengthens the hook.

In the inoperative position of lower holder part, used for detaching the hook from the axle, an outer surface of the hook wall is projected some upward relative to a lower surface of the movable element in its released state, thereby preventing the lower holder part from spontaneous falling out of the upper holder part in the inoperative position, and providing for a need for dislocating the movable element upward in order to detach the lower holder part from the upper holder part as well as in order to put the hook on the axle. Such design of the hook is novelty allowing the hook to fulfill the part of the functions fulfilled by the ball keeper in the existing laryngoscope.

The holder fastening means include a holder locking means preventing the lower holder part from spontaneous dislocation relative to the upper holder part in the operation position. The holder locking means comprises a detent means preventing the lower holder part from a spontaneous rotation around the axle in the operation position, including: a detent recess located on an inner side of at least one of the lateral walls; and a ball springy detent mounted at an insertion portion edge opposite to the edge, at which the hook is disposed, and having a ball adapted to enter the detent recess in the operation position and to come out from the detent recess in the inoperative position.

According to the present invention, the intubation laryngoscope is provided with a blade fastening means designed for detachable fastening the detachable blade to the lower holder part, and a blade locking means preventing the detachable blade from disengagement from the lower holder part in the operation position. The blade fastening means include: a blade socket located at the blade proximal end portion and opened from a proximal side of the detachable blade; and a fastening protrusion of the lower holder part protruding distally of the insertion portion of lower holder part, adapted to inserting into the blade socket and having external configuration corresponding to an inner configuration of the blade socket to maximally restrict a displacement of the detachable blade relative to the lower holder part after inserting the fastening protrusion into the blade socket.

After full inserting the fastening protrusion into the blade socket in the inoperative position and subsequent setting the operation position of the lower holder part, the blade proximal portion is extended some proximally of a proximal edge of the upper holder part and comprises at least one lug, which is located on a proximal end of the blade proximal portion proximally of a proximal edge of the upper holder part so that an extreme lug distal surface contacts with an extreme proximal surface of the upper holder part thereby fulfilling a function of the blade locking means preventing the detachable blade from displacement distally of the lower holder part in the operation position. As long as the holder locking means, specifically the ball springy detent, hold the lower holder part in the operation position, the blade locking means in the form of the lug hold the detachable blade from disengagement from the lower holder part.

There is also an additional blade locking means designed for preventing the detachable blade from spontaneous detachment from the lower holder part in the inoperative position and providing for a need for some operator's effort to disconnect the detachable blade and the lower holder part in the inoperative position. The additional blade locking means is made as a latch appliance including a resilient leg located on the blade proximal portion and having a latch opening, as well as a latch projection located on the lower holder part and adapted to entering the latch opening, when the fastening protrusion of lower holder part is inserted into said blade socket.

The offered blade locking means in the form of the lug is novelty, which along with the mentioned blade fastening means and additional blade locking means allow obtaining the extreme simple and inexpensive blade design in the form of a single plastic piece.

In version embodiments, the axle is disposed at an acute angle up to a zero angle to a plane passing through a longitudinal axis of said handle and the distal direction of detachable blade extension, and internal surfaces of the two lateral walls are disposed at an acute angle up to a right angle to the mentioned plane. Specifically in one version, the angle of axle disposition is zero and the disposition angle of the internal surfaces of two lateral walls approximately is right. The use of these angles is novelty allowing maximal reduction of the requirements for the strength of the lower holder part, specifically for the strength of the hook, due to the redistribution of loading the elements of the lower holder part. As a result, the capability of lower holder part manufacture of plastic, having sufficient strength, arises. In another version, the angle of axle disposition is equal to 45° and the disposition angle of the internal surfaces of two lateral walls also is equal to 45°. This version is characterized with some decreased hook strength but has improved the transversal fixation of the lower holder part relative to the upper holder part in the operative position.

The offered lower holder part allows the following versions of the illumination means:
1) the illumination means include batteries located within the handle, the light source located in the upper holder part, and a light guide, which in the operation position is located in the lower holder part between the light source and a distal face of the lower holder part;
2) the illumination means include batteries located within the handle and electrically connected with the movable element, the light source located in the distal end of the lower holder part, and a lead wire located in the lower holder part and connecting the light source with an electric contact, which is disposed on an upper surface of the insertion portion of lower holder part and in the operation position contacting with the movable element;
3) the illumination means, including batteries and the light source, fully disposed in the lower holder part. The latter version is novelty, allowing considerable simplifying and price reducing the design of the handle and the upper holder part.

### BRIEF DESCRIPTION OF THE DRAWINGS

FIGS. 1 to 3 show the intubation laryngoscopes and their components of Prior Art, wherein:

FIG. 1 shows a holder lower part immovably affixed to a detachable blade.

FIG. 2 shows a holder upper part immovably affixed to a laryngoscope handle.

FIG. 3 shows the laryngoscope assembly of company “Welch Allyn”.

The drawings presented in the following relate to the present invention.

FIGS. 4 to 8 show a version of an offered laryngoscope having a light source arranged in an upper holder part, as well as a hook of a lower holder part enveloping an axle of the upper holder part from above, from below and from a proximal side in an operation position, wherein:

FIG. 4 shows a subassembly of the holder upper part immovably affixed to a laryngoscope handle and including the light source.

FIG. 5 shows a subassembly of the lower holder part including a hook and a light guide, to be movably affixed to the holder upper part, and adapted to detachable connection with a laryngoscope blade.

FIG. 6 shows the detachable blade depleted of any components of illumination means.

FIG. 7 shows an external view of a laryngoscope assembly.

FIG. 8 shows a cross-sectional view of the offered laryngoscope.

FIGS. 9 to 17 show another version of the offered laryngoscope having a light source arranged in an upper holder part, as well as a hook of a lower holder part enveloping an axle of the upper holder part from above, from a distal side and from a proximal side in an operation position, wherein:

FIG. 9 shows a subassembly of the lower holder part including the light guide and the hook.

FIG. 10 shows a subassembly of the upper holder part comprising the light source.

FIG. 11 shows the detachable blade.

FIG. 12 shows an external view of a laryngoscope assembly.

FIG. 13 shows a cross-sectional view of the offered laryngoscope in an operation position.

FIG. 14 shows a cross-sectional view of the offered laryngoscope in an inoperative position.

FIGS. 15 to 17 show three stages of attaching the detachable blade to a holder.

FIGS. 18 to 32 show another version of an offered laryngoscope having a light source arranged in a holder lower part, as well as a hook of a lower holder part enveloping an axle of the upper holder part from above, from a distal side and from a proximal side in an operation position, wherein:

FIG. 18 shows a detachable blade.

FIG. 19 shows a subassembly of the upper holder part adapted for transmitting electrical energy from batteries housed in a handle to the light source.

FIG. 20 shows a subassembly of the lower holder part.

FIG. 21 shows an external view of a laryngoscope assembly.

FIG. 22 shows an external view of an illuminating insertion appliance including components of an electrical circuit of the laryngoscope illumination means.

FIG. 23 shows a ball springy detent.

FIG. 24 shows a cross-sectional view of the illuminating insertion appliance.

FIG. 25 shows the holder lower part.

FIG. 26 shows a close-up view of a lower holder part subassembly including the illuminating insertion appliance.

FIGS. 27 to 30 show a version embodiment of the lower holder part subassembly, wherein the components of an electrical circuit are incorporated immediately into a holder plastic body.

FIG. 31 shows a cross-sectional view of the laryngoscope assembly in an operation position.

FIG. 32 shows a cross-sectional view of the laryngoscope assembly in an inoperative position.

FIGS. 33 to 38 show another version of the offered laryngoscope with a modified arrangement of an insertion portion of a lower holder part, wherein:

FIG. 33 shows a detachable blade.

FIG. 34 shows an upper holder part.

FIG. 35 shows a subassembly of the lower holder part.

FIG. 36 shows an external view of the laryngoscope in an operation position.

FIG. 37 shows an external view of the laryngoscope in an inoperative position.

FIG. 38 shows a cross-sectional view of the laryngoscope in the operation position.

FIGS. 39 to 41 show a laryngoscope version having a modified arrangement of an insertion portion of a lower holder part and a non-detachable affixation of the lower holder part to a blade, wherein:

FIG. 39 shows a subassembly of a handle and an upper holder part.

FIG. 40 shows a detachable unit including the lower holder part and the blade.

FIG. 41 shows an external view of the laryngoscope in an operation position.

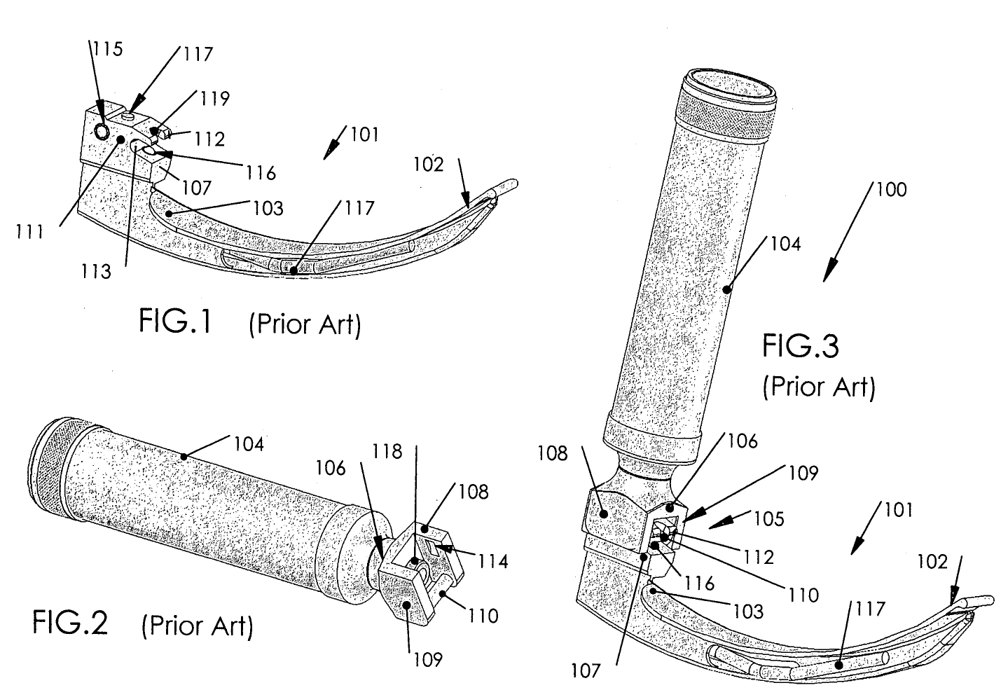

### DETAILED DESCRIPTION OF THE INVENTION

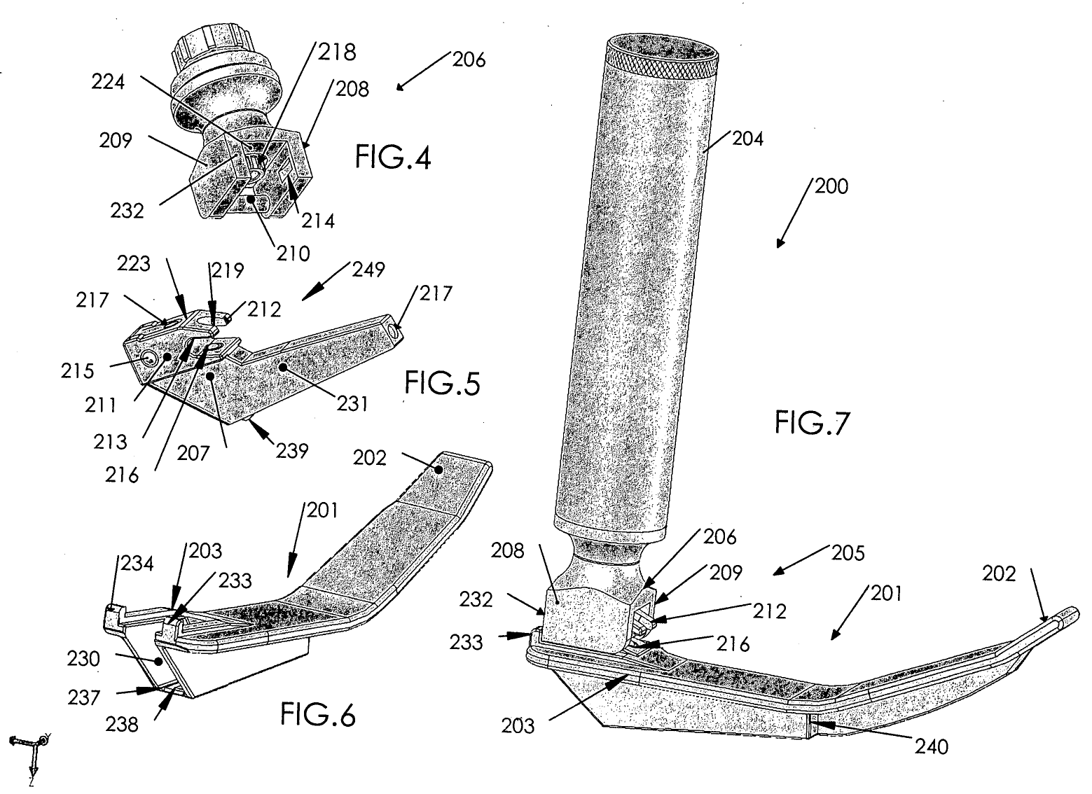

The explanation of the present invention is offered with references made to the attached drawings in FIGS. 4 to 41.

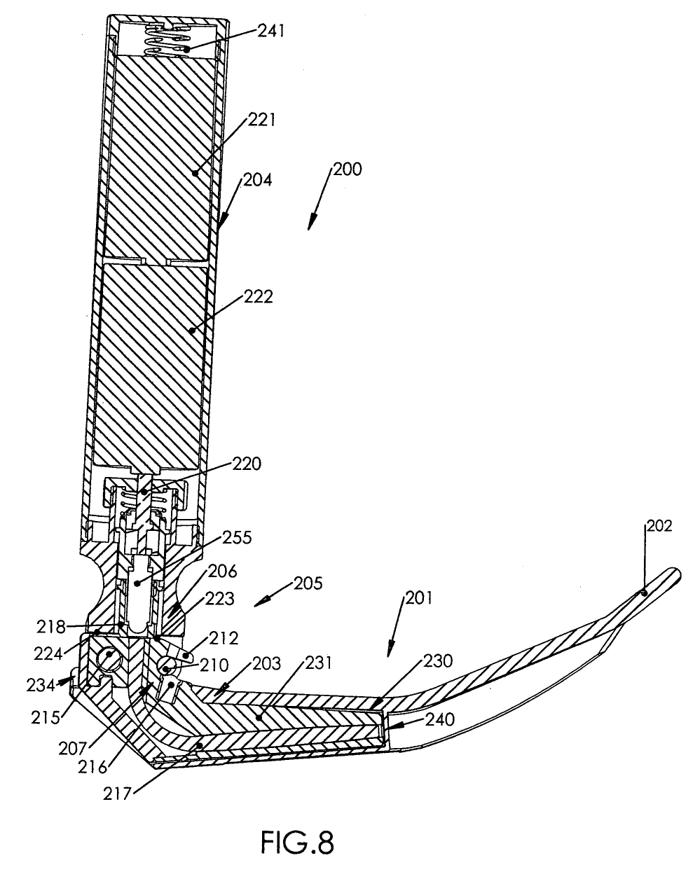

The drawings in FIGS. 4 to 8 show intubation laryngoscope 200 comprising handle 204 and detachable blade 201, extending from a lower end of handle 204 in a distal direction and having blade proximal end portion 203 and blade distal end portion 202 designed to expose a tracheal entrance in order to insert an endothracheal tube therein. This blade is one of several blades having different sizes and forming a laryngoscope blade set. There is also holder 205 designed for fastening blade proximal end portion 203 to the lower end of handle 204. Holder 205 consists of upper holder part 206 constantly immovably affixed to the lower end of handle 204 and lower holder part 207 to be movably fastened to the upper holder part and adapted for detachable connection with blade proximal end portion 203. Laryngoscope 200 is provided with an illumination means (FIG. 8) designed to illuminate a zone of the endotracheal tube insertion and including light source 255 housed in upper holder part 206 and connectable with batteries 221, 222 through turning means 220. The latter is designed for turning on/off an illumination light. The illumination means also comprises light guide 217 disposed in lower holder part 207 and along with the latter forming lower holder part assembly 249.

Such lower holder part is a novelty allowing using the same single lower holder part for each blade of the blade set, thereby providing a decrease in the price of laryngoscope in comparison with the prior art, where each blade has its own lower holder part. Specifically, this novelty provides substantial design simplification and decrease in price of the detachable blades due to eliminating the lower holder part and the light guide from the blade design. At the same time, the offered lower holder part allows to maintain the movable connection of upper and lower holder parts for turning on/off the illumination light. Moreover, there is a capability of the easy laryngoscope adaptation to operation with various blade kinds by modifying only the single lower holder part without modifying the handle and upper holder part.

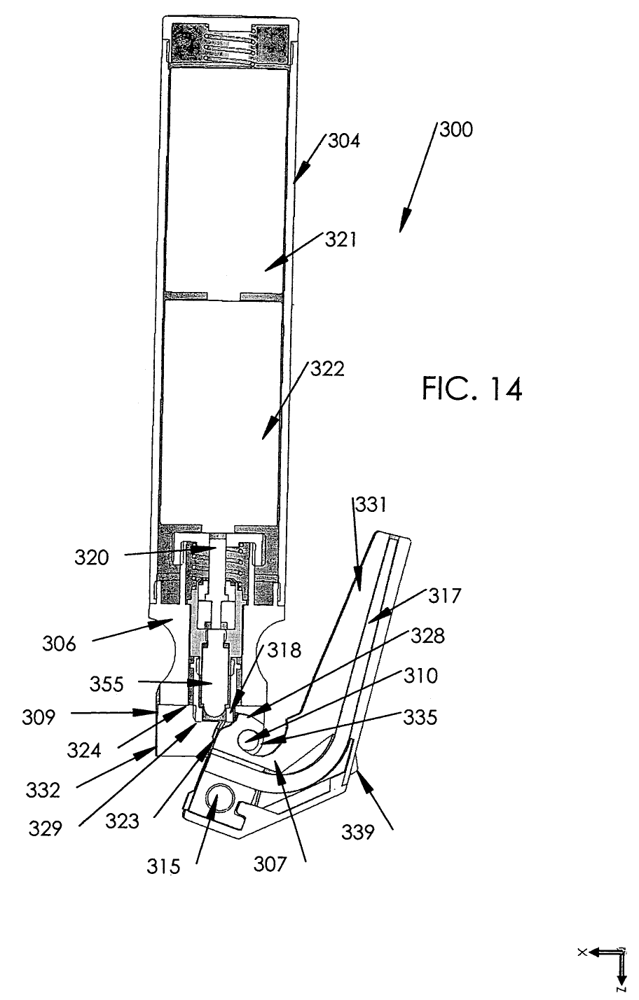

Lower holder part 207 has an operation position and an inoperative position relative to upper holder part 206. The operation position, shown in FIGS. 7, 8, allows an intubation execution and is characterized by substantially immovable fixation of lower holder part 207 to upper holder part 206, substantially immovable fixation of detachable blade 201 to lower holder part 207, and a switched-on position of turning means 220 to provide the illumination light. The inoperative position, obtained by rotating the lower holder part counter-clockwise and demonstrated subsequently with identical design in FIG. 14, allows attachment/detachment of detachable blade 201 to/from lower holder part 207, provides a capability of detachment of lower holder part 207 from upper holder part 206, and is characterized by a switched-off position of turning means 220 disconnecting light source 255 and battery 222 and thereby turning off the illumination light. Upper surface 223 of insertion portion 211 and a longitudinal axis of handle 204 are disposed substantially at right angle in the operation position, and at angle more than 90° in the inoperative position.

Lower holder part 207 is detachably fastened to upper holder part 206 by a holder fastening means. The latter includes two lateral walls 208, 209 of upper holder part 206 spaced some distance apart and protruding downward. Axle 210 is mounted between lateral walls 208, 209 at a distal edge of upper holder part 206. Lower holder part 207 has insertion portion 211 to be inserted between lateral walls 208, 209 with a small gap, and having hook 212 formed by groove 213, disposed at a distal edge of insertion portion 211 and adapted to putting on axle 210 with a capability of rotating the lower holder part 207 around axle 210 to set one of the above mentioned positions of the lower holder part. There is also a holder locking means preventing lower holder part 207 from spontaneous dislocation relative to upper holder part 206 in the operation position.

Turning means 220 includes movable element 218 disposed in upper holder part 206 between lateral walls 208, 209 at some distance from axle 210, protruding downward from inner upper surface 224 of upper holder part 206 in the inoperative position, and interacting with lower holder part 207 so that in the operation position of lower holder part 207 the movable element 218 is dislocated upward by pressure of lower holder part 207 (FIG. 8), thereby turning on the illumination light. In the inoperative position, movable element 218 is released by lower holder part 207 that leads to returning downward the movable element and thereby to turning off the illumination light.

The above mentioned holder locking means comprises a detent means preventing lower holder part 207 from a spontaneous rotation around axle 210 in the operation position. The detent means includes detent recess 214 located on an inner side of at least one of lateral walls 208, 209, and ball springy detent 215 mounted at a proximal edge of insertion portion 211 opposite to the edge, at which hook 212 is disposed, and having a ball adapted to enter detent recess 214 in the operation position and to come out from detent recess 214 in the inoperative position.

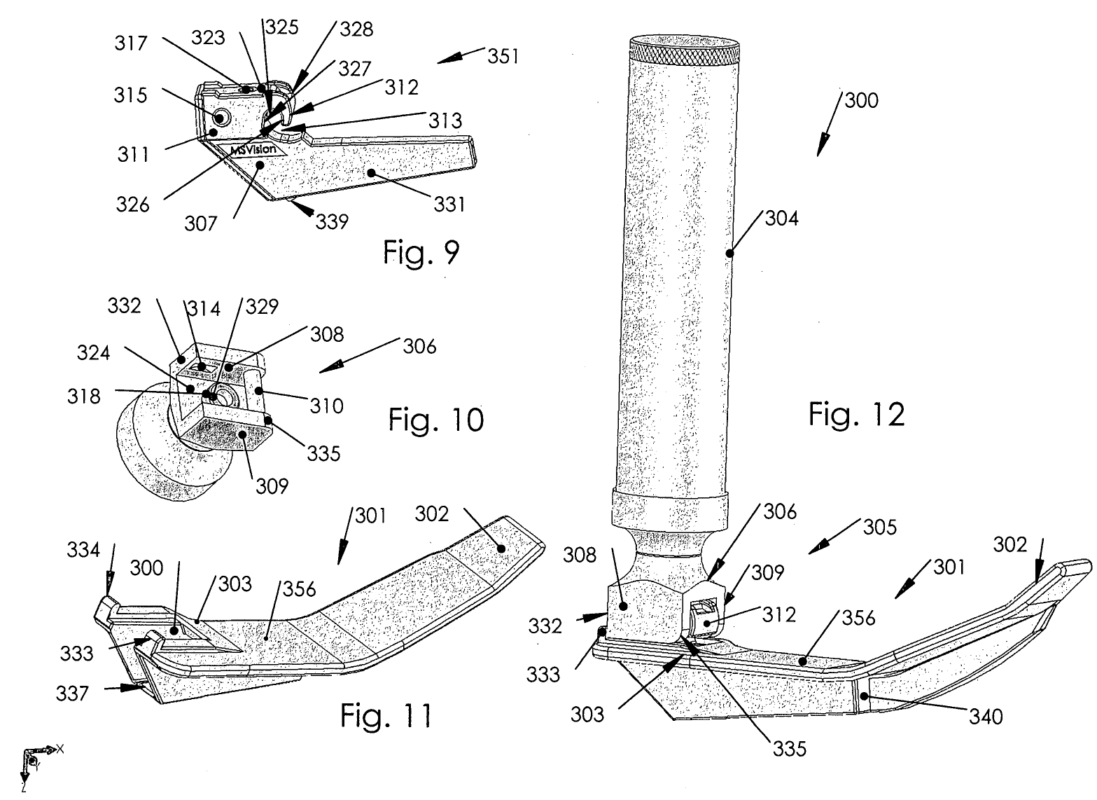

FIGS. 9 to 17 show offered laryngoscope version 300 wherein hook 312 of lower holder part 307 has a modified form. Laryngoscopes 200 and 300 have many elements having identical design and operation and designated by figures with the same two last numerals. Therefore, the description of version 300 is mainly devoted to its distinctions.

As distinct from above version, hook 312 (FIGS. 9, 13) is formed by groove 313 having U-shaped upper portion 325 directed downward with its open side, so that inner walls 326, 327 of U-shaped upper portion 325 are disposed substantially in parallel with a longitudinal axis of handle 304 and interact with axle 310 thereby preventing lower holder part 307 from dislocating in a distal and proximal directions relative to upper holder part 306 in the operation position. Inner walls 326, 327 of U-shaped upper portion 325 of hook 312 also are disposed substantially at a right angle to upper surface 323 of insertion portion 311 of lower holder part 307 with a permissible deviation ±30° from the right angle. In other words, in the operation position, inner walls of hook 312 envelop axle 310 from above, from distal side and from proximal side, while in the prior art (laryngoscope versions 100) and in version 200 the hook inner walls envelop the axle from above, from below and from proximal side. This modification of hook groove 313 is novelty preventing lower holder part 307 from a dislocation relative to upper holder part 306 in the proximal and distal directions (in the prior art the hook groove restricted the dislocation only in the distal direction) and allowing the groove inner walls to fulfill the function of ball keeper 216, shown in FIG. 5. As a result, ball keeper 216 and upper window 219 used for the ball keeper mounting are eliminated in lower holder part 307 that strengthens the hook and the lower holder part and reduces the laryngoscope price.

In inoperative position of lower holder part 307 (FIG. 14), used for disconnecting hook 312 and axle 310, upper outer surface 328 of a hook wall is projected some upward relative to lower surface 329 of movable element 318 in its released state. In other words, in the inoperative position, surfaces 328 and 329 are disposed with some overlap in the vertical direction. As a result, lower holder part 307 is prevented from spontaneous falling out of upper holder part 306 in the inoperative position. To detach the lower holder part 307 from upper holder part 306 as well as to put hook 312 on axle 310 movable element 318 must be dislocated upward by operator.

Laryngoscope 300 has a blade fastening means designed for detachable fastening the detachable blade 301 to lower holder part 307, as well as a blade locking means preventing detachable blade 301 from disengagement from lower holder part 307 in the operation position. The blade fastening means includes blade socket 330 made as a cavity, located below blade upper operating wall 356 at blade proximal end portion 303 and opened from a proximal side, as well as fastening protrusion 331 of lower holder part 307 protruding distally of insertion portion 311, adapted to inserting into blade socket 330 and having external configuration corresponding to an inner configuration of blade socket 330 to maximally restrict a displacement of detachable blade 301 relative to lower holder part 307 after inserting fastening protrusion 331 into blade socket 330. Specifically, fastening protrusion 331 and blade socket 300 have a substantially rectangular form in a transversal section, providing restricting the very important transverse dimensions of blade socket 300 and fastening protrusion 331 as well as preventing twisting the detachable blade 301 during the intubation procedure. Fastening protrusion 331 serves also as a support of detachable blade 301 during the intubation procedure. As a result, the substantial decrease in the requirements for blade strength and the corresponding decrease in the thickness of blade walls and blade weight are achieved. In a longitudinal section (FIG. 13), blade socket 330 and fastening protrusion 331 have a conical form easing the insertion of fastening protrusion 331 into blade socket and decreasing their vertical dimensions at their distal portions.

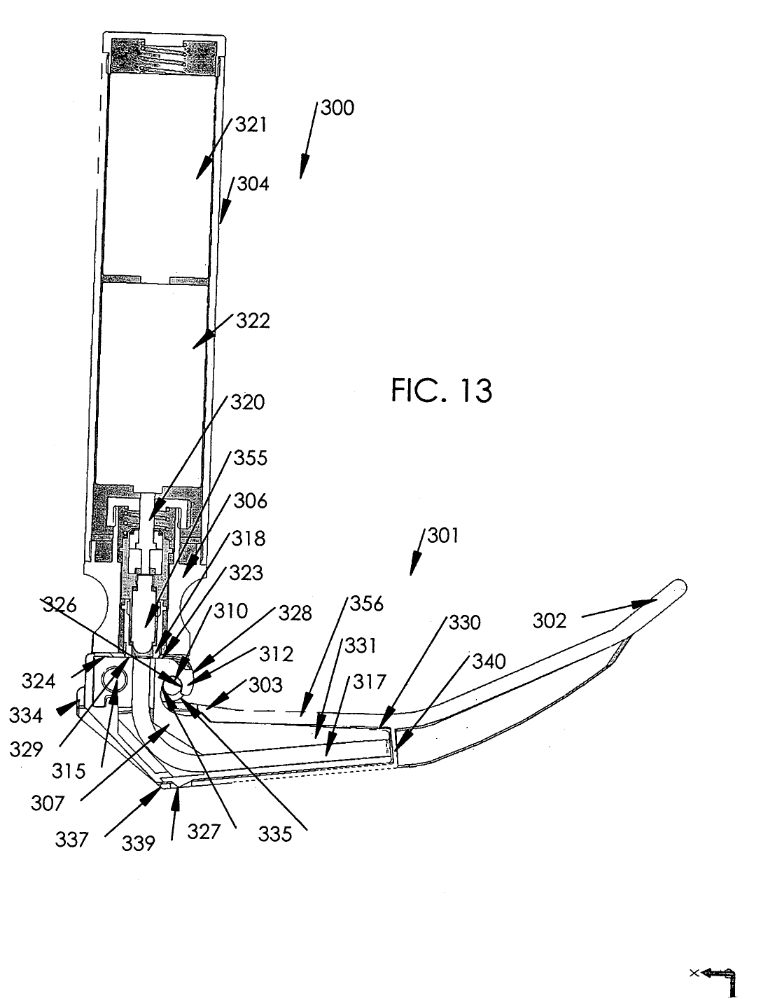

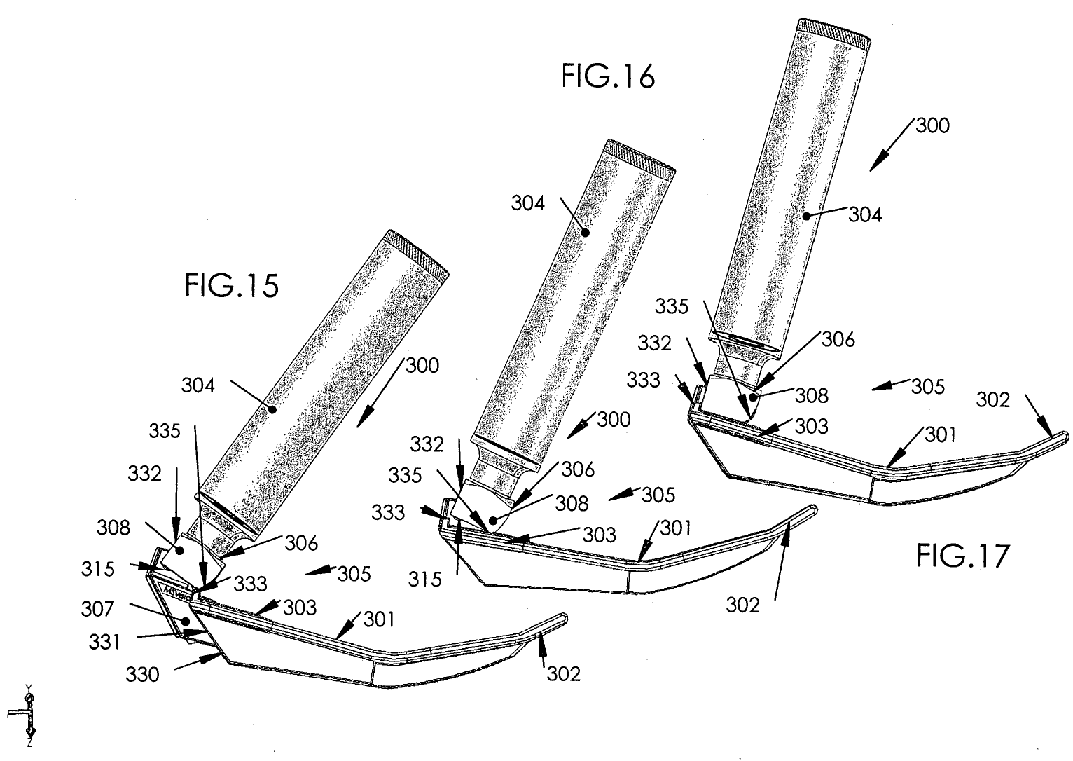

The blade locking means includes lugs 333, 334 disposed on the blade proximal end. FIGS. 15 to 17 show the process of fastening detachable blade 301 to laryngoscope holder 305. In the first stage of the fastening (FIG. 15), fastening protrusion 331 of lower holder part 307 is partly inserted into blade socket 330 in the inoperative position of lower holder part 307, therewith the conical form of fastening protrusion 331 and blade socket 330 allow free passing the lugs 333, 334 past lower edge 335 of upper holder part 306. In the second stage (FIG. 16), fastening protrusion 331 is fully inserted into blade socket 330 in the inoperative position of lower holder part 307. In the third stage (FIG. 17), lower holder part 307 along with detachable blade 301 is transferred into the operation position by rotating them clockwise around axle 310. In the operation position (FIGS. 12, 13, 17), blade proximal end portion 303 is extended some proximally of proximal edge 332 of upper holder part 306 so that lugs 333, 334 are located proximally of proximal edge 332 of upper holder part 306 thereby preventing detachable blade 301 from displacement distally of lower holder part 307 as long as lower holder part 307 is in the operation position. In order to detach detachable blade 301 from lower holder part 307, the latter must be transferred into the inoperative position (FIG. 16) by its rotating counter-clockwise.

There is an additional blade locking means designed for preventing detachable blade 301 from spontaneous detachment from lower holder part 307 in the inoperative position. This means (FIG. 13) is made as latch appliance 336 including resilient leg 337 located on blade proximal end portion 303 and having latch opening 338, as well as latch projection 339 located on lower holder part 307 and adapted to entering latch opening 338, when fastening protrusion 331 of lower holder part 307 is inserted into blade socket 330. To detach detachable blade 301 from lower holder part 307 in the inoperative position, some operator's effort directed distally is necessary.

The offered blade locking means in the form of lugs 333, 334 is novelty, which along with the mentioned blade fastening means and additional blade locking means allow obtaining the extreme simple and inexpensive design of detachable blade 301 in the form of a single plastic piece.

The presented above laryngoscope versions 200 and 300 are provided with the illumination means including batteries 221, 222 (FIG. 3) and 321, 322 (FIGS. 13, 14) located within handles 204 and 304 respectively, light sources 255 and 355 located in upper holder parts 206 and 306 respectively, and light guides 217 and 317 located in lower holder parts 207 and 307 respectively between the light source and a distal face of the lower holder part in the operation position of the lower holder part. Therewith the light source may be a conventional lamp or a light emitting device (LED).

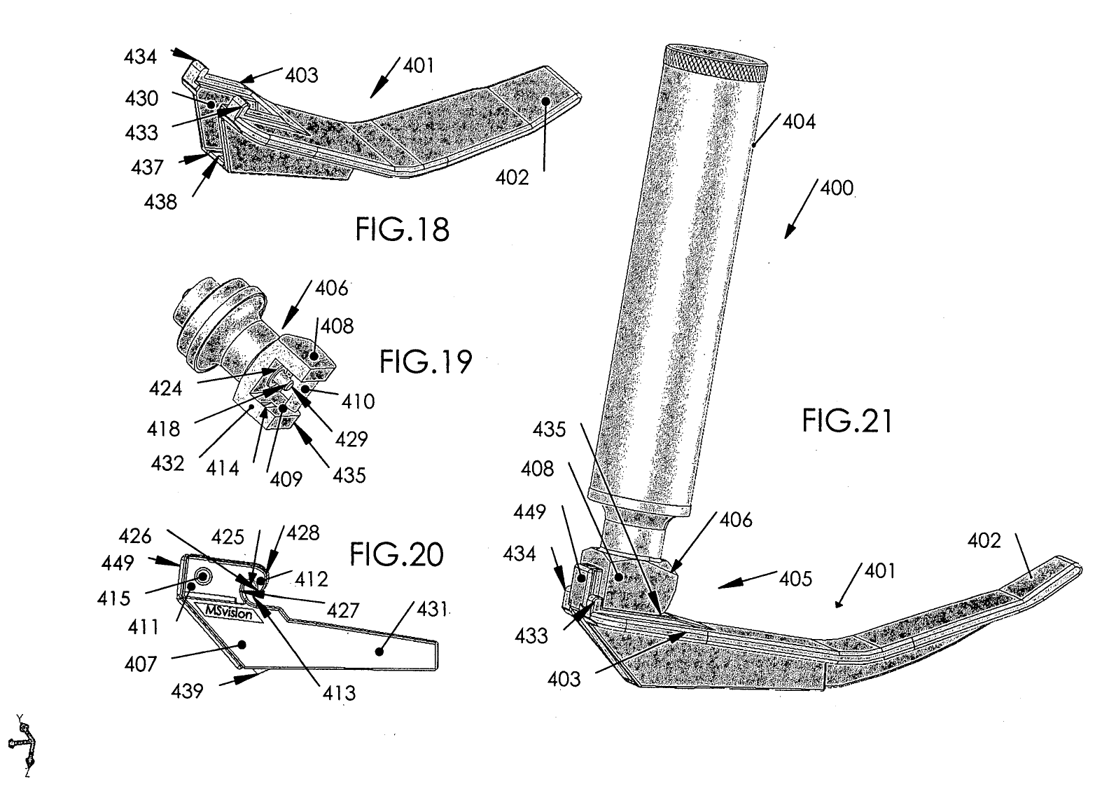

Version embodiment 400 of the offered intubation laryngoscope, shown in FIGS. 18 to 32, and above laryngoscopes 200, 300 include many elements having identical design and operation and designated by figures with the same two last numerals. Therefore, the following description of version 400 is mainly restricted with its distinctions.

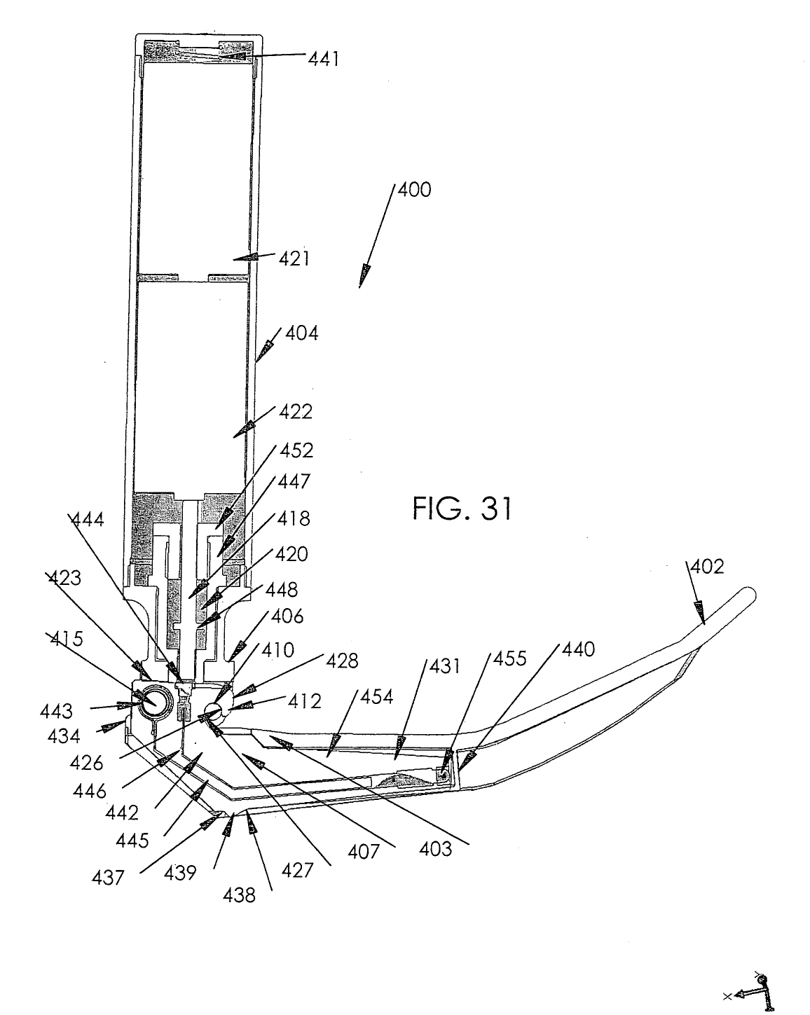

As distinct from the above laryngoscopes 200 and 300, laryngoscope 400 has the illumination means (FIG. 31) including batteries 421, 422 located within handle 404, and light source 455 in the form of lamp or LED located in a distal end of lower holder part 407. The batteries and light source are connectable through first lead wire 446 and second lead wire 445 located in lower holder part 407. Therewith, first lead wire 446 connects light source 455 immediately with electrical contact 444, which is disposed at upper surface 423 of lower holder part 407 and in the operation position of lower holder part 407 contacting with movable element 418 of turning means 420. Movable element 418, insulated from upper holder part 406 by movable element keeper 447 with its cover 452 made of plastic, in turn contacts with a positive pole of battery 422. Second lead wire 445 connects light source 455 with metal contacting bushing 443, which in the operation position of lower holder part 407 is connected with a negative pole of battery 421 through ball springy detent 415, metallic upper holder part 406, metallic handle 404 and spring 441. Electrical contact 444 and contacting bushing 443 are contacting members allowing said lead wires 445, 446 to interact with turning means 420.

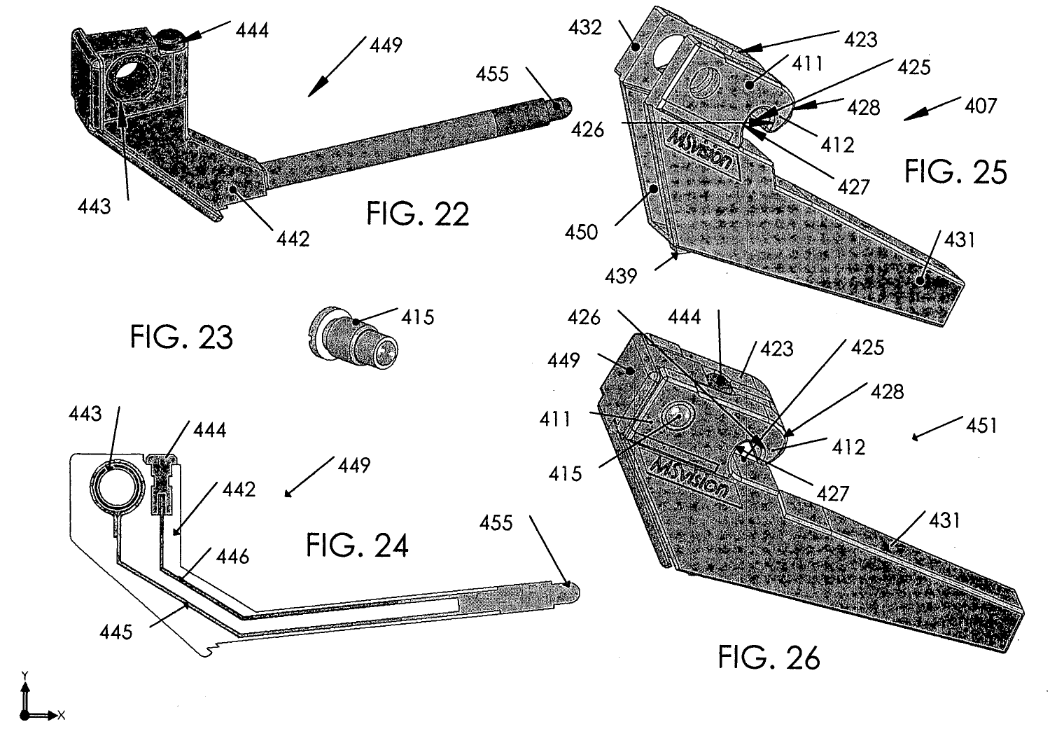

In version embodiment (FIGS. 22 to 26), electrical contact 444, contacting bushing 443, lead wires 445, 446 and light source 455 are incorporated into plastic housing 442 forming the illuminating insertion appliance 449. The latter is inserted into holder socket 450 of lower holder part 407 and fastened to it by ball springy detent 415, thereby forming lower holder part assembly 451. As a result, assembly 451 consists of the illuminating insertion appliance 449, whose housing 442 is made of plastic, and lower holder part 407 made of metal or plastic.

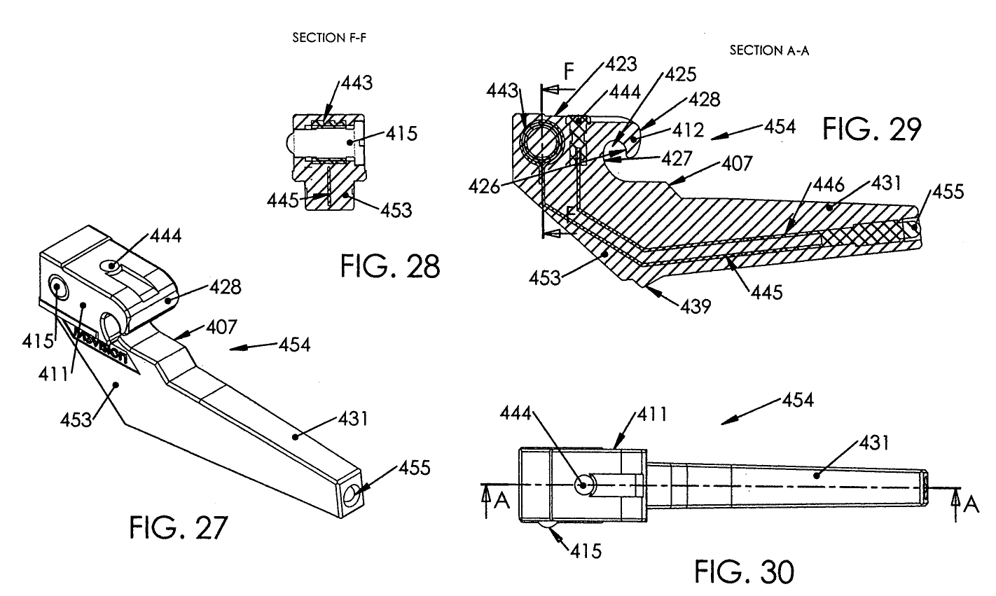

In another version embodiment, shown in FIGS. 27 to 30, contacting bushing 443, electrical contact 444, light source 455 and lead wires 445, 446 are incorporated immediately into plastic body 453 of lower holder part 407, thereby forming a subassembly 454 of the lower holder part.

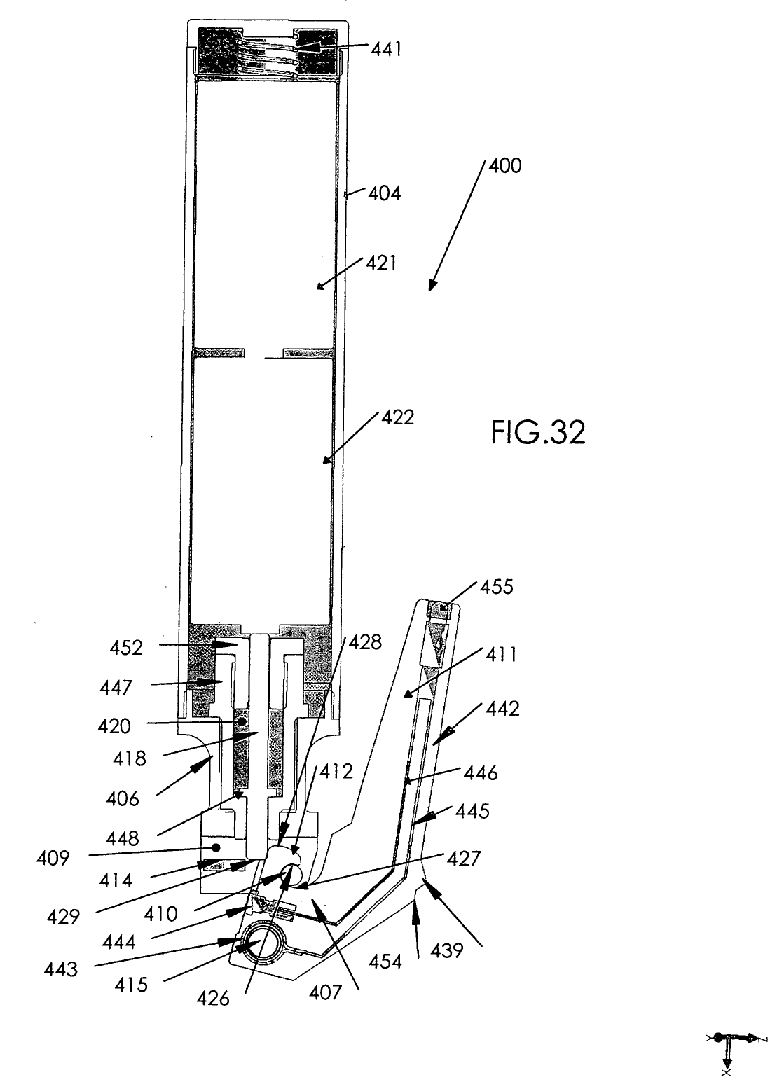

In inoperative position of lower holder part (FIG. 32), the contacts between electrical contact 444 and movable element 418 as well as between ball springy detent 415 and upper holder part 406 are interrupted and the illumination light is turned off.

The batteries 421, 422 are constantly squeezed between spring 441 and movable element 418 thereby providing a reliable electrical contact between components 444, 418, 422, 421, 404, 406 in the operation position of lower holder part 407 (FIG. 32). In the inoperative position (FIG. 32), spring 441 continues pressing onto movable element 418 due to that the latter protrudes upward from cover 452 of movable element keeper 447. This protrusion is provided with the ratio of the lengths of movable element 418 and keeper 447, as well as disposition of limiting flange 448 on movable element 418. As a result, in the inoperative position, spring 441 holds movable element 418 in an extreme lower position needed for preventing lower holder part 407 from spontaneous falling out of upper holder part 406. Thus, batteries 421, 422 are contacting with movable element 418 in any position. The offered mechanism of interacting spring 441, batteries 421, 422 and movable element 418 is novelty allowing simplifying the design of turning means 420 due to using only one spring 441 both as a battery holding means and as a spring of turning means 420, as well as enhancing a reliability of the electrical circuit due to decreasing the number of disconnectable contacts, while in the identical mechanism in the prior art at least two springs as well as the disconnecting contact of the movable element and battery are used. In FIGS. 31, 32 is used holder subassembly 454, shown in FIGS. 27 to 30 and having a plastic housing 453 eliminating a short circuit between movable element 448 and lower holder part 407 in the inoperative position (FIG. 32).

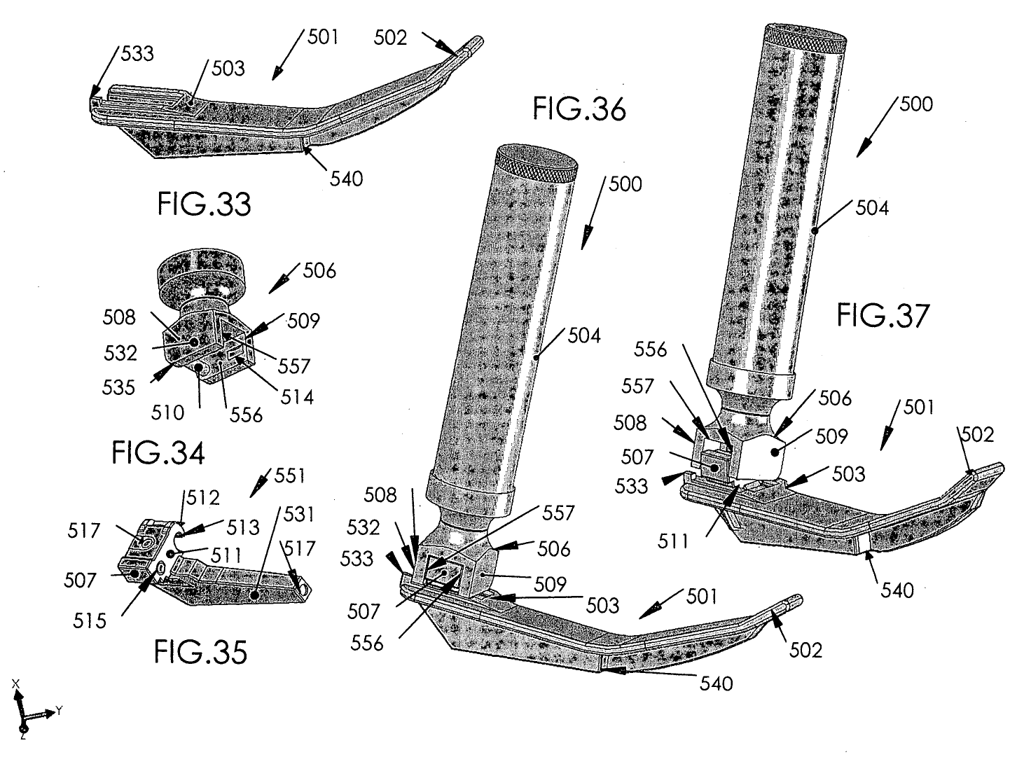

Version embodiment 500 of the offered intubation laryngoscope, shown in FIGS. 33 to 38, and above laryngoscopes 200, 300, 400 include many elements having identical design and operation and designated by figures with the same two last numerals. Therefore, the description of version 500 is mainly devoted to its distinctions.

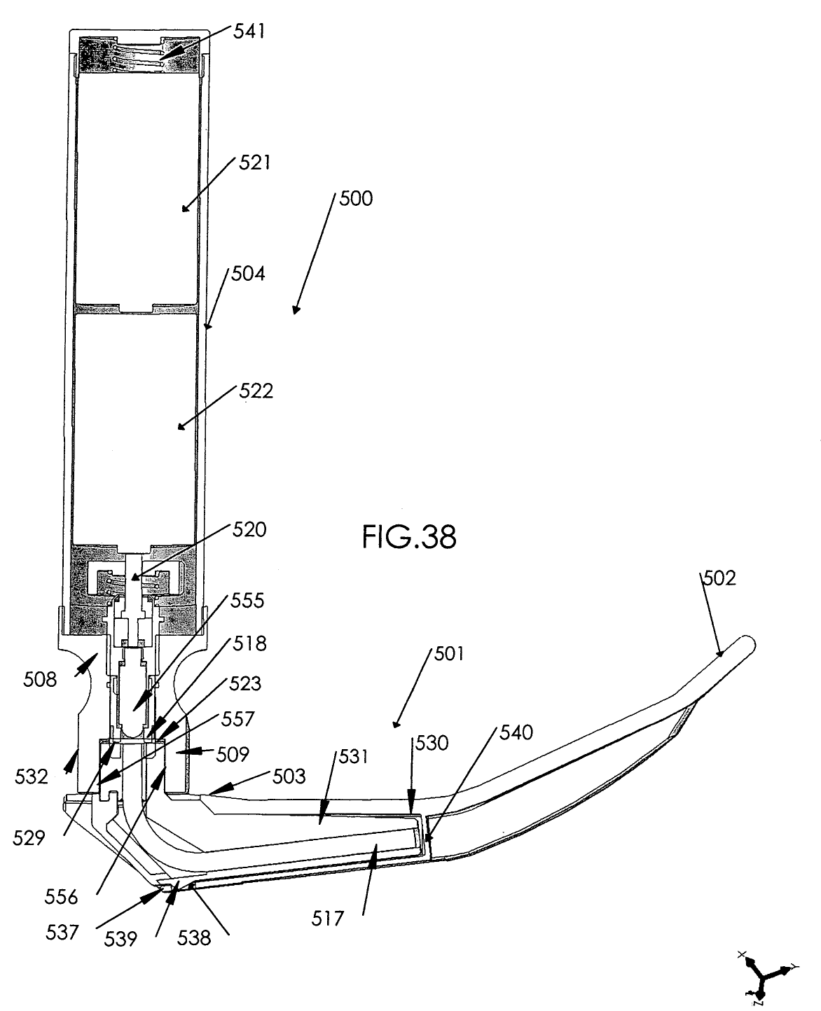

In version embodiment 500 (FIGS. 33 to 38), axle 510 of upper holder part 506 is disposed in parallel to a middle plane passing through a longitudinal axis of handle 504 and a distal direction of extending the detachable blade 50. The middle plane is a secant plane in the cross-sectional view in FIG. 38. Internal surfaces 556, 557 of two lateral walls 508, 509, respectively, are disposed at right angle to the middle plane, while in the prior art and in the above laryngoscopes 200, 300, 400 the axle of upper holder part is disposed at right angle to the middle plane and two lateral walls of the upper holder part have internal surfaces disposed in parallel to the middle plane. In accordance with new arrangement of axles 510 and lateral walls 508, 509, the groove 513 of hook 512 is disposed in parallel to the middle plane, and insertion portion 511 of lower holder part 507 is disposed transversely to the middle plane (FIG. 35). As a result, the load of lower holder part 507, specifically in the form of a torque acting in the middle plane and arising during the intubation procedure, to a great extent is applied to lateral walls 508, 509, instead of the hook in the prior art. This leads to substantial increase of the resistance power of the lower holder part due to that walls 508, 509 are thicker and stronger than hook 512. In inoperative position, shown in FIG. 37, upper holder part 506 along with handle 504 is some rotated around axle 510 in a plane perpendicular to the middle plane, thereby obviating obstacle for passing lug 533 and allowing putting the detachable blade 501 on lower holder part 507. After returning in the operation position (FIG. 36), lug 553 is disposed proximally of wall 508 thereby preventing detachable blade 501 from spontaneous detachment from lower holder part 507 in the operation position.

The offered arrangement of axle 510, hook grove 513, insertion part 511 and lateral walls 508, 509 is novelty allowing maximal reduction of the requirements for the strength of lower holder part 507, specifically for the strength of hook 512, due to the redistribution of the load between the elements of the lower holder part. As a result, the capability of manufacturing the plastic lower holder part, having sufficient strength, is achieved.

In another versions of laryngoscope 500 (not shown), the angle between axle 510 and the middle plane is acute, for example it is equal to 45°, with corresponding changes of the disposition of another elements such as 511, 513, 508, 509 relative to the middle plane. Therewith, the less is this acute angle the more is the resistance power of the lower holder part. The maximal resistance power of the lower holder part is achieved when the mentioned angle is equal to zero, i.e. in the version, shown in FIGS. 34 to 38. The necessity of some increasing the mentioned angle more than zero may be caused with the need for some enhancing the operation effectiveness of the detent means, specifically ball spring detent 515.

All above presented laryngoscope versions 200, 300, 400, 500 have the illumination system, wherein all components of the illumination means are disposed in a laryngoscope subassembly consisting of the handle and the both holder parts.

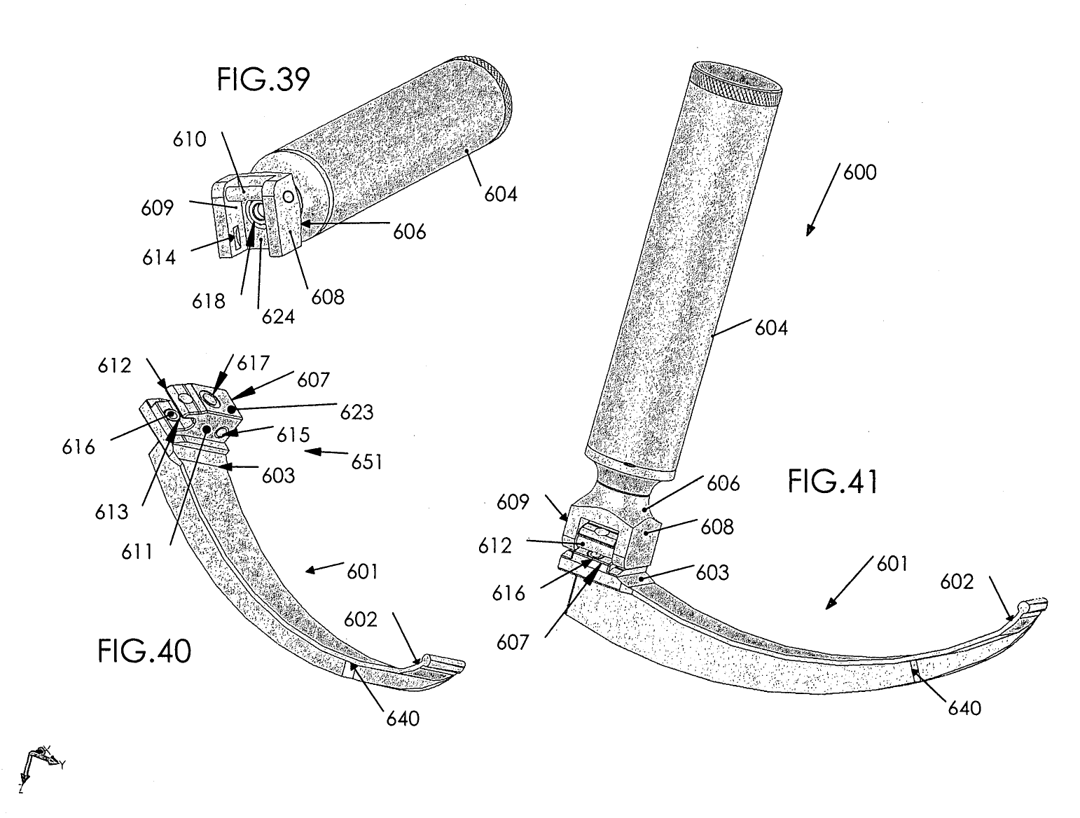

The modified arrangement of the axle and other elements of the upper and lower holder parts, shown in FIGS. 33 to 38, in principle, may be also applied to the laryngoscopes, which are beyond the offered laryngoscopes having the lower holder part detachable from the blade. Specifically, FIGS. 39 to 41 show laryngoscope 600, wherein lower holder part 607 is constantly attached to bade 601 and along with the blade is detachable from upper holder part 606 identically to laryngoscope 100 of the prior art. However, the arrangement of axle 610, lateral walls 608, 609, insertion portion 611 and groove 613 of hook 612 is identical to one of above laryngoscope 500. Laryngoscopes 500 and 600 have the same two last numerals of the identical element designations. This allows applying the description of the peculiarity of laryngoscope 500 also to laryngoscope 600. Specifically, the offered arrangement of axle 610 and the other above pointed elements allows considerable reduction in the requirements for the strength of lower holder part 607, specifically for the strength of hook 612, due to the redistribution of the load between the elements of lower holder part 607. As a result, the capability of manufacturing plastic lower holder part 607, as well as a plastic unit of lower holder part 607 and blade 601 as a whole, having sufficient strength, is achieved.

1. An intubation laryngoscope comprising
a handle,
at least one detachable blade, extending from a lower end of said handle in a distal direction and having
a blade distal end portion designed to expose a tracheal entrance in order to insert an endothracheal tube therein,
a blade proximal end portion,
a holder designed for fastening said blade proximal end portion to said handle lower end and including
an upper holder part located at a lower end of said handle and immovable relative to it,
and a lower holder part
to be movably fastened to said upper holder part
and adapted for detachable connection with said blade proximal end portion,
an illumination means designed to illuminate a zone of said endotracheal tube insertion and provided with a light source and a turning means for turning on/off an illumination light.

2. The intubation laryngoscope of claim 1, wherein said lower holder part has an operation position and an inoperative position relative to said upper holder part, therewith
said operation position allows an intubation execution and is characterized by
substantially immovable fixation of said lower holder part to said upper holder part,
substantially immovable fixation of said detachable blade to said lower holder part,
and a switched-on position of said turning means to provide said illumination light,
and said inoperative position
allows attachment/detachment of said detachable blade to/from said lower holder part,
provides a possibility for detachment of said lower holder part from said upper holder part,
and is characterized by a switched-off position of said turning means.

3. The intubation laryngoscope of claim 2, wherein there are
a holder fastening means designed for movable fastening said lower holder part to said upper holder part and including
two lateral walls of said upper holder part spaced some distance apart and protruding downward,
an axle mounted between said lateral walls at an edge of said upper holder part,
an insertion portion of said lower holder part to be inserted between said lateral walls with a small gap,
a hook disposed at an edge of said insertion portion of lower holder part and adapted to putting on said axle with a capability of rotating said lower holder part around said axle to set one of said positions of said lower holder part relative to said upper holder part,
a holder locking means preventing said lower holder part from spontaneous dislocation relative to said upper holder part in said operation position.

4. The intubation laryngoscope of claim 3, wherein an upper surface of said insertion portion of lower holder part and a longitudinal axis of said handle are disposed substantially at right angle in said operation position, and at angle more than 90° in said inoperative position.

5. The intubation laryngoscope of claim 3, wherein said turning means includes a movable element movably mounted on said upper holder part between said lateral walls at some distance from said axle, being under acting a movable element spring, and interacting with said lower holder part so that in said operation position of said lower holder part said movable element is dislocated upward by pressure of said lower holder part thereby turning on said illumination light, and in said inoperative position said movable element is released by said lower holder part that leads to returning downward said movable element by action of a movable element spring and thereby to turning off said illumination light.

6. The intubation laryngoscope of claim 5, wherein said holder locking means comprises a detent means preventing said lower holder part from a spontaneous rotation around said axle in said operation position, including
a detent recess located on an inner side of at least one of said lateral walls,
and a ball springy detent mounted at an edge of said insertion portion opposite to said edge, at which said hook is disposed, and having a ball adapted to enter said detent recess in said operation position and to come out from said detent recess in said inoperative position.

7. The intubation laryngoscope of claim 6, wherein said hook is formed as a groove having a U-shaped upper portion whose inner walls in said operation position of lower holder part envelop said axle from above and from two lateral sides thereby preventing said lower holder part from dislocating in a lateral directions relative to said upper holder part in said operation position.

8. The intubation laryngoscope of claim 7, wherein, in said inoperative position of lower holder part used for attaching/detaching said hook to/from said axle and characterized with enveloping of said axle by said hook inner walls from above, from below and from one of two lateral sides, an upper outer surface of a hook wall is projected some upward relative to a lower surface of said movable element in its released state, forming some overlap of said hook upper surface and said movable element lower surface, thereby preventing said lower holder part from spontaneous falling out of said upper holder part in said inoperative position, and requiring some dislocating said movable element upward in order to detach said hook from said axle as well as in order to put said hook on said axle.

9. The intubation laryngoscope of claim 8, wherein said axle and a middle plane, passing through a longitudinal axis of said handle and a distal direction of said detachable blade extension, are disposed at an angle to one another lying in the range of 0 to 90°, therewith said angle determines a distribution of an operation load between said hook and said two lateral walls, so that the less is said angle the more is a load share applied to said lateral walls and the more is a resistance power of said lower holder part as a whole.

10. The intubation laryngoscope of claim 9, wherein said angle is equal 90° and said two lateral sides are a distal and proximal sides, therefore said inner walls of hook grove in said operation position of lower holder part envelop said axle from above, from said distal side and from said proximal side thereby preventing said lower holder part from dislocating in a said distal and proximal directions relative to said upper holder part in said operation position, and in said inoperative position, used for attaching/detaching said hook to/from said axle, the latter is enveloped by said hook inner walls from above, from below and from said proximal side, therewith said angle value provides a minimal resistance power of said lower holder part.

11. The intubation laryngoscope of claim 9, wherein said angle is equal 0° and said two lateral sides are a left-hand and right-hand sides (relative to the operator's position), therefore said inner walls of hook grove in said operation position of lower holder part envelop said axle from above, from said left-hand side and from said right-side side thereby preventing said lower holder part from dislocating in said left-hand and right-hand directions relative to said upper holder part in said operation position, and in said inoperative position, used for attaching/detaching said hook to/from said axle, the latter is enveloped by said hook inner walls from above, from below and from one of said left-hand and right-hand sides, therewith said angle value provides a maximal resistance power of said lower holder part.

12. The intubation laryngoscope of claim 9, wherein said angle is equal to 45°, providing a mean resistance power of said lower holder part.

13. The intubation laryngoscope of claim 9, wherein there is a blade fastening means designed for detachable fastening said detachable blade to said holder, and a blade locking means preventing said detachable blade from disengagement from said holder in said operation position.

14. The intubation laryngoscope of claim 13, wherein said blade fastening means include
a blade socket made as a cavity, located at said blade proximal end portion below an upper surface of said blade proximal end portion, and opened from a proximal side of said detachable blade, and
a fastening protrusion of said lower holder part protruding distally of said insertion portion of lower holder part and adapted to inserting into said blade socket,
and an external configuration of said fastening protrusion and an internal configuration of said blade socket after inserting said fastening protrusion into said blade socket are corresponding to one another and formed to maximally restrict twisting and bending said detachable blade as well as its dislocation relative to said lower holder part during an intubation procedure, resulting in substantial decrease of the requirements for a blade strength and a corresponding decrease of a thickness of blade walls and blade weight.

15. The intubation laryngoscope of claim 14, wherein said configuration of blade socket and fastening protrusion has a rectangular outline in a transversal section, preventing said detachable blade from rotating around said fastening protrusion and twisting, as well as a substantially conical outline in a longitudinal section easing inserting said insertion protrusion into said blade socket and including an upper line consisting of a proximal horizontal portion and a distal inclined portion providing an optimal load distribution between said detachable blade and said insertion protrusion, when said insertion portion serves as a support of said detachable blade during the intubation procedure.

16. The intubation laryngoscope of claim 14, wherein said blade locking means comprises at least one lug located on a proximal end of said blade proximal portion, protruding upward, and disposed proximally of a proximal edge of said upper holder part in said operation position of lower holder part, so that said proximal edge of upper holder part prevents said lug along with said detachable blade from displacement distally, resulting in a need to transfer said lower holder part into said inoperative position in order to detach said detachable blade from said lower holder part.

17. The intubation laryngoscope of claim 16, wherein there is an additional blade locking means preventing said detachable blade from spontaneous detachment from said lower holder part in said inoperative position and requiring some operator's effort to detach said detachable blade from said lower holder part in said inoperative position.

18. The intubation laryngoscope of claim 17, wherein said additional blade locking means is made as a latch appliance including a resilient leg located on said blade proximal portion and having a latch opening, as well as a latch projection located on said lower holder part and adapted to entering said latch opening, when said fastening protrusion of lower holder part is inserted into said blade socket.

19. The intubation laryngoscope of claim 14, wherein said illumination means is fully located in a laryngoscope unit consisting of said handle and said both holder parts including said fastening protrusion of lower holder part, and said blade socket has at least a transparent distal face transmitting distally said illumination light emitted from a distal end of said fastening protrusion.

20. The intubation laryngoscope of claim 19, wherein said illumination means include batteries located within said handle, said light source located in said upper holder part, and a light guide housed in said lower holder part and, in said operation position of lower holder part, disposed between said light source and said transparent distal face.

21. The intubation laryngoscope of claim 19, wherein said illumination means include batteries located within said handle and electrically contacting with said movable element in said operation position of lower holder part, said light source located in a distal end portion of lower holder part, and two lead wires located in said lower holder part, therewith first lead wire connects said light source with an electrical contact disposed at said upper surface of insertion portion of lower holder part and in said operation position contacting with a battery positive pole through said movable element, and second lead wire in said operation position of lower holder part connects said light source with a negative battery pole through a contacting bushing, said ball springy detent housed in said contacting bushing and said upper holder part; and in said inoperative position of lower holder part the contacts between said electrical contact and movable element as well as between said ball springy detent and upper holder part are interrupted and the illumination light is turned off.

22. The intubation laryngoscope of claim 21, wherein said electrical contact, contacting bushing, lead wires and light source are incorporated into a plastic housing thereby forming an illuminating insertion appliance as a separated unit to be inserted into a holder socket of said lower holder part and fixed therein by said ball springy detent, therewith said lower holder part may be made of metal or plastic.

23. The intubation laryngoscope of claim 21, wherein said electrical contact, contacting bushing, lead wires and light source are incorporated immediately into a body of said lower holder part made of plastic.

24. The intubation laryngoscope of claim 21, wherein said movable element has a permanent contact with said battery positive pole both in said operation position and in inoperative position of lower holder part, and said illumination means has only one spring located above said batteries and serving both as a battery holding means and as a spring of said turning means permanently acting onto said movable element.

25. An intubation laryngoscope comprising
a handle,
at least one detachable blade extending from a lower end of said handle in a distal direction and having
a blade distal end portion designed to expose a tracheal entrance in order to insert an endothracheal tube therein,
a blade proximal end portion,
a holder designed for fastening said blade proximal end portion to said handle lower end and including
an upper holder part immovably affixed to a lower end of said handle,
a lower holder part fastened to said blade proximal end portion, and movably fastened to said upper holder part,
a holder fastening means designed for movable fastening said lower holder part to said upper holder part, including
two lateral walls of said upper holder part spaced some distance apart and protruding downward,
an axle mounted between said lateral walls at an edge of said upper holder part,
a hook
disposed at an edge of said lower holder part,
adapted to putting on said axle with a capability of rotating said lower holder part around said axle to set an operation position of said lower holder part relative to said upper holder part, wherein an intubation execution is possible, and an inoperative position, wherein said intubation execution is impossible,
and made as a grove in said lower holder part having a U-shaped upper portion, whose walls envelop said axle from above and from two lateral sides in said operation position, thereby preventing said lower holder part from dislocating in a lateral directions relative to said upper holder part in said operation position; and envelop said axle from above, from below and from one of said two lateral sides in said inoperative position keeping free another lateral side for putting said hook on said axle as well as for detaching said hook from said axle,
an illumination means designed to illuminate a zone of said endotracheal tube insertion and provided with a light source and a turning means for turning on/off an illumination light.

26. The intubation laryngoscope of claim 25, wherein there is a detent means preventing said lower holder part from a spontaneous rotation around said axle in said operation position, including
a detent recess located on an inner side of at least one of said lateral walls,
and a ball springy detent mounted at an edge of said lower holder part oppositely to a disposition of said hook and having a ball adapted to enter said detent recess in said operation position and to come out from said detent recess in said inoperative position.

27. The intubation laryngoscope of claim 26, wherein said turning means includes a movable element disposed in said upper holder part between said lateral walls at some distance from said axle and interacting with said lower holder part so that in said operation position of said lower holder part said movable element is dislocated upward by pressure of said lower holder part thereby turning on said illumination light, and in said inoperative position said movable element is released by said lower holder part that leads to returning downward said movable element by a spring action and thereby to turning off said illumination light.

28. The intubation laryngoscope of claim 27, wherein, in said inoperative position of lower holder part used for putting said hook on said axle, an upper outer surface of said hook is projected some upward relative to a lower surface of said movable element in its released state, forming some overlap between said hook upper outer surface and said movable element lower surface thereby preventing said lower holder part from spontaneous falling out of said upper holder part in said inoperative position, and requiring some dislocating said movable element upward in order to detach said lower holder part from said upper holder part as well as in order to put said hook on said axle.

29. The intubation laryngoscope of claim 28, wherein said axle and a middle plane, passing through a longitudinal axis of said handle and a distal direction of said detachable blade extension, are disposed at an angle to one another lying in the range of 0 to 90°, therewith said angle determines a distribution of an operation load between said hook and said two lateral walls, so that the less is said angle the more is a load share applied to said lateral walls and consequently the more is a resistance power of said lower holder part as a whole.

30. The intubation laryngoscope of claim 29, wherein said angle is equal 90° and said two lateral sides are a distal and proximal sides, and consequently said inner walls of hook grove in said operation position of lower holder part envelop said axle from above, from said distal side and from said proximal side thereby preventing said lower holder part from dislocating in a said distal and proximal directions relative to said upper holder part in said operation position, and in said inoperative position, used for attaching/detaching said hook to/from said axle, the latter is enveloped by said hook inner walls from above, from below and from said proximal side, therewith said angle value provides a minimal resistance power of said lower holder part.

31. The intubation laryngoscope of claim 29, wherein said angle is equal 0° and said two lateral sides are a left-hand and right-hand sides (relative to the operator's position), therefore said inner walls of hook grove in said operation position of lower holder part envelop said axle from above, from said left-hand side and from said right-side side thereby preventing said lower holder part from dislocating in said left-hand and right-hand directions relative to said upper holder part in said operation position, and in said inoperative position, used for attaching/detaching said hook to/from said axle, the latter is enveloped by said hook inner walls from above, from below and from one of said left-hand and right-hand sides, therewith said angle value provides a maximal resistance power of said lower holder part.

32. The intubation laryngoscope of claim 29, wherein said angle is equal to 45°, providing a mean resistance power of said lower holder part.

33. The intubation laryngoscope of claim 29, wherein said detachable blade is immovably affixed to said lower holder part and is detachable from said upper holder part along with said lower holder part.

34. The intubation laryngoscope of claim 33, wherein there is a blade set including several said detachable blades of different sizes, therewith each said detachable blade of said blade set is provided with its own said lower holder part.

35. The intubation laryngoscope of claim 34, wherein said illumination means include batteries located within said handle, said light source located in said upper holder part, and a light guide disposed partly in said lower holder part and partly on said detachable blade.

36. The intubation laryngoscope of claim 34, wherein said illumination means include: batteries located within said handle and electrically connected with said movable element, said light source located on said detachable blade, and at least one electric wire located partly in said lower holder part and partly on said detachable blade and connecting said light source with an electric contact, which is disposed on said lower holder part and in said operation position contacting with said movable element.

37. The intubation laryngoscope of claim 29, wherein said detachable blade is detachable from said lower holder part and thereby from said handle.

38. The intubation laryngoscope of claim 37, wherein there is a blade set, including several said detachable blades of different sizes, and only one common said lower holder part used for each detachable blade of said blade set.

39. The intubation laryngoscope of claim 38, wherein there is a blade fastening means designed for detachable fastening said detachable blade to said holder, and a blade locking means preventing said detachable blade from disengagement from said holder in said operation position.

40. The intubation laryngoscope of claim 39, wherein said blade fastening means include
a blade socket made as a cavity, located at said blade proximal end portion below an upper surface of said blade proximal end portion, and opened from a proximal side of said detachable blade, and
a fastening protrusion of said lower holder part protruding distally of said insertion portion of lower holder part and adapted to inserting into said blade socket,
and an external configuration of said fastening protrusion and an internal configuration of said blade socket after inserting said fastening protrusion into said blade socket are corresponding to one another and formed to maximally restrict twisting and bending said detachable blade as well as its dislocation relative to said lower holder part during an intubation procedure, resulting in substantial decrease of the requirements for a blade strength and a corresponding decrease of a thickness of blade walls and blade weight.

41. The intubation laryngoscope of claim 40, wherein said blade locking means comprises at least one lug located on a proximal end of said blade proximal portion, protruding upward, and disposed proximally of a proximal edge of said upper holder part in said operation position of lower holder part, so that said proximal edge of upper holder part prevents said lug along with said detachable blade from displacement distally, resulting in a need to transfer said lower holder part into said inoperative position in order to detach said detachable blade from said lower holder part.

42. The intubation laryngoscope of claim 41, wherein there is an additional blade locking means preventing said detachable blade from spontaneous detachment from said lower holder part in said inoperative position and requiring some operator's effort to detach said detachable blade from said lower holder part in said inoperative position.

43. The intubation laryngoscope of claim 42, wherein said additional blade locking means is made as a latch appliance including a resilient leg located on said blade proximal portion and having a latch opening, as well as a latch projection located on said lower holder part and adapted to entering said latch opening, when said fastening protrusion of lower holder part is inserted into said blade socket.

44. The intubation laryngoscope of claim 40, wherein said illumination means is fully located in a laryngoscope unit consisting of said handle and said both holder parts including said fastening protrusion of lower holder part, and said blade socket has at least a transparent distal face transmitting distally said illumination light emitted from a distal end of said fastening protrusion.

45. The intubation laryngoscope of claim 44, wherein said illumination means include batteries located within said handle, said light source located in said upper holder part, and a light guide housed in said lower holder part and, in said operation position of lower holder part, disposed between said light source and said transparent distal face.

46. The intubation laryngoscope of claim 44, wherein said illumination means include batteries located within said handle and electrically contacting with said movable element in said operation position of lower holder part, said light source located in a distal end portion of lower holder part, and two lead wires located in said lower holder part, therewith first lead wire connects said light source with an electrical contact disposed at said upper surface of insertion portion of lower holder part and in said operation position contacting with a battery positive pole through said movable element, and second lead wire in said operation position of lower holder part connects said light source with a negative battery pole through a contacting bushing, said ball springy detent housed in said contacting bushing and said upper holder part; and in said inoperative position of lower holder part the contacts between said electrical contact and movable element as well as between said ball springy detent and upper holder part are interrupted and the illumination light is turned off.

47. An intubation laryngoscope comprising
a handle,
at least one detachable blade extending from a lower end of said handle in a distal direction and having
a blade distal end portion designed to expose a tracheal entrance in order to insert an endothracheal tube therein, p2 a blade proximal end portion,
a holder designed for fastening said blade proximal end portion to said handle and including
an upper holder part immovably affixed to a lower end of said handle,
and a lower holder part
detachably fastened to said blade proximal end portion,
and detachably fastened to said upper holder part by a holder fastening means, which include
two lateral walls of said upper holder part spaced some distance apart and protruding downward,
an axle mounted between said lateral walls at an edge of said upper holder part,
a hook disposed in said lower holder part, adapted to putting on said axle of upper holder part with a capability of rotating said lower holder part around said axle to set an operation position of said lower holder part relative to said upper holder part, wherein an intubation execution is possible, and an inoperative position, wherein said intubation execution is impossible,
made as a grove in said lower holder part having a U-shaped upper portion, whose walls envelop said axle from above and from two lateral sides in said operation position, thereby preventing said lower holder part from dislocating in a lateral directions relative to said upper holder part in said operation position; and envelop said axle from above, from below and from one of said two lateral sides in said inoperative position keeping free another lateral side for putting said hook on said axle as well as for detaching said hook from said axle,
an illumination means designed to illuminate a zone of said endotracheal tube insertion and provided with a light source and a switch for turning on/off an illumination light.

48. The intubation laryngoscope of claim 47, wherein there is a detent means preventing said lower holder part from a spontaneous rotation around said axle in said operation position, including
a detent recess located on an inner side of at least one of said lateral walls,
and a ball springy detent mounted at a lower holder part edge opposite to said edge, at which said hook is disposed, and having a ball adapted to enter said detent recess in said operation position and to come out from said detent recess in said inoperative position.

49. The intubation laryngoscope of claim 48, wherein said axle and a middle plane, passing through a longitudinal axis of said handle and a distal direction of said detachable blade extension, are disposed at an angle to one another lying in the range of 0 to 90°, therewith said angle determines a distribution of an operation load between said hook and said two lateral walls, so that the less is said angle the more is a load share applied to said lateral walls and consequently the more is a resistance power of said lower holder part as a whole.

50. The intubation laryngoscope of claim 49, wherein said angle is equal 90° and said two lateral sides are a distal and proximal sides, and consequently said inner walls of hook grove in said operation position of lower holder part envelop said axle from above, from said distal side and from said proximal side thereby preventing said lower holder part from dislocating in said distal and proximal directions relative to said upper holder part in said operation position, and in said inoperative position, used for attaching/detaching said hook to/from said axle, the latter is enveloped by said hook inner walls from above, from below and from said proximal side, therewith said angle equal to 90° provides a minimal resistance power of said lower holder part.

51. The intubation laryngoscope of claim 49, wherein said angle is equal 0° and said two lateral sides are a left-hand and right-hand sides (relative to the operator's position), therefore said inner walls of hook grove in said operation position of lower holder part envelop said axle from above, from said left-hand side and from said right-side side thereby preventing said lower holder part from dislocating in said left-hand and right-hand directions relative to said upper holder part in said operation position; and in said inoperative position, used for attaching/detaching said hook to/from said axle, the latter is enveloped by said hook inner walls from above, from below and from one of said left-hand and right-hand sides, therewith said angle equal to 0° provides a maximal resistance power of said lower holder part.

52. An intubation laryngoscope comprising
a handle,
at least one detachable blade extending from a lower end of said handle in a distal direction and having
a blade distal end portion designed to expose a tracheal entrance in order to insert an endothracheal tube therein,
a blade proximal end portion,
a holder designed for fastening said blade proximal end portion to said handle lower end and including
an upper holder part immovably affixed to said handle lower end,
and a lower holder part fastened to said blade proximal end portion, and movably fastened to said upper holder part by a holder fastening means, which include
two lateral walls of said upper holder part spaced some distance apart and protruding downward,
an axle mounted between said lateral walls at an edge of said upper holder part and disposed at an angle less than 90° to a middle plane, passing through a longitudinal axis of said handle and a distal direction of said detachable blade extension,
a hook
disposed at an edge of said lower holder part,
adapted to putting on said axle with a capability of rotating said lower holder part around said axle in a plane, which is substantially normal to said distal direction, in order to set an operation position of said lower holder part relative to said upper holder part, wherein an intubation execution is possible, and an inoperative position, wherein said intubation execution is impossible,
an illumination means designed to illuminate a zone of said endotracheal tube insertion and provided with a light source and a switch for turning on/off an illumination light.

53. The intubation laryngoscope of claim 52, wherein said angle is equal 0°, that leads to a redistribution of an operation load from said hook to said two lateral walls and thereby provides a considerable increase of a resistance power of said lower holder part.

54. The intubation laryngoscope of claim 52, wherein said angle is equal to 45°, providing a mean increase of a resistance power of said lower holder part.

55. The intubation laryngoscope of claim 52, wherein there is a detent means preventing said lower holder part from a spontaneous rotation around said axle in said operation position and including
a detent recess located on an inner side of at least one of said lateral walls,
and a ball springy detent mounted at a lower holder part edge opposite to said edge, at which said hook is disposed, and having a ball adapted to enter said detent recess in said operation position and to come out from said detent recess in said inoperative position.

56. The intubation laryngoscope of claim 52, wherein said lower holder part is immovably fastened to said blade proximal end portion and said detachable blade is detachable from said upper holder part along with said lower holder part.

57. The intubation laryngoscope of claim 56, wherein there is a blade set including several said detachable blades of different sizes, therewith each said detachable blade of said blade set is provided with its own said lower holder part.

58. The intubation laryngoscope of claim 52, wherein said lower holder part is detachably fastened to said blade proximal end portion and said detachable blade is detachable from said lower holder part and thereby from said handle.

59. The intubation laryngoscope of claim 58, wherein there is a blade set, including several said detachable blades of different sizes, and only single common said lower holder part used for each detachable blade of said blade set.

60. The intubation laryngoscope of claim 59, wherein there is a blade fastening means designed for detachable fastening said detachable blade to said holder, and a blade locking means preventing said detachable blade from disengagement from said holder in said operation position.

61. The intubation laryngoscope of claim 60, wherein said blade fastening means include
a blade socket made as a cavity, located at said blade proximal end portion below an upper surface of said blade proximal end portion, and opened from a proximal side of said detachable blade, and
a fastening protrusion of said lower holder part protruding distally of said insertion portion of lower holder part and adapted to inserting into said blade socket,
and an external configuration of said fastening protrusion and an internal configuration of said blade socket after inserting said fastening protrusion into said blade socket are corresponding to one another and formed to maximally restrict twisting and bending said detachable blade as well as its dislocation relative to said lower holder part during an intubation procedure, resulting in substantial decrease of the requirements for a blade strength and a corresponding decrease of a thickness of blade walls and blade weight.

62. The intubation laryngoscope of claim 61, wherein said configuration of blade socket and fastening protrusion has a rectangular outline in a transversal section, preventing said detachable blade from rotating around said fastening protrusion and twisting, as well as a substantially conical outline in a longitudinal section easing inserting said insertion protrusion into said blade socket and including an upper line consisting of a proximal horizontal portion and a distal inclined portion providing an optimal load distribution between said detachable blade and said insertion protrusion, when said insertion portion serves as a support of said detachable blade during the intubation procedure

63. The intubation laryngoscope of claim 62, wherein said blade locking means comprises at least one lug located on a proximal end of said blade proximal portion, protruding upward, and disposed proximally of a proximal edge of said upper holder part in said operation position of lower holder part, so that said proximal edge of upper holder part prevents said lug along with said detachable blade from displacement distally, resulting in a need to transfer said lower holder part into said inoperative position in order to detach said detachable blade from said lower holder part.

64. The intubation laryngoscope of claim 63, wherein there is an additional blade locking means preventing said detachable blade from spontaneous detachment from said lower holder part in said inoperative position and requiring some operator's effort to detach said detachable blade from said lower holder part in said inoperative position.

65. The intubation laryngoscope of claim 64, wherein said additional blade locking means is made as a latch appliance including a resilient leg located on said blade proximal portion and having a latch opening, as well as a latch projection located on said lower holder part and adapted to entering said latch opening, when said fastening protrusion of lower holder part is inserted into said blade socket.

66. The intubation laryngoscope of claim 52, wherein said hook is formed as a groove having a U-shaped upper portion whose inner walls in said operation position of lower holder part envelop said axle from above and from two lateral sides thereby preventing said lower holder part from dislocating in a lateral directions relative to said upper holder part in said operation position.

67. An intubation laryngoscope comprising
a handle,
at least one detachable blade, extending from a lower end of said handle in a distal direction and having
a blade distal end portion designed to expose a tracheal entrance in order to insert an endothracheal tube therein,
a blade proximal end portion,
a holder designed for fastening said blade proximal end portion to said handle lower end
an illumination means designed to illuminate a zone of said endotracheal tube insertion, fully located in a laryngoscope unit consisting of said handle and said holder, and including
batteries located within said handle,
a light source located in a distal end portion of said holder,
a turning means for turning on/off an illumination light,
two lead wires located in said holder and connecting said light source with said batteries through said turning means, therewith said lead wires are provided with contacting members for interacting with said turning means.
